(19) United States
(12) Patent Application Publication
(10) Pub. No.: US 2010/0056934 A1
(43) Pub. Date: Mar. 4, 2010

(54) APPARATUS AND METHOD FOR MEASURING PULSE WAVE

(75) Inventors: Hyeonsung Cho, Daejeon-city (KR); Joochan Sohn, Daejeon-city (KR); Jaehong Kim, Daejeon-city (KR); Chankyu Park, Daejeon-city (KR); Yunkoo Chung, Daejeon-city (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon-city (KR)

(21) Appl. No.: 12/512,359

(22) Filed: Jul. 30, 2009

(30) Foreign Application Priority Data
Aug. 28, 2008 (KR) 10-2008-0084761

(51) Int. Cl. A61B 5/02

(52) U.S. Cl. 600/502

(57) ABSTRACT

A pulse wave measuring apparatus disposes a plurality of light emitting elements and a plurality of light receiving elements in a line, measures pulse wave signals at locations where the light emitting elements and the light receiving elements are located, when the pulse wave measuring apparatus is worn on a wrist of the person to be examined, and determines an optimal pulse wave signal among the plurality of measured pulse wave signals to measure pulse waves. An optimal pulse wave can be detected from an optimal pulse wave signal that is detected by a combination of the light emitting element and the light receiving element that are adjacent to a blood vessel of a person to be examined.

### RELATED APPLICATIONS

The present application claims priority to Korean Patent Application Serial Number 10-2008-0084761, filed on Aug. 28, 2008, the entirety of which is hereby incorporated by reference.

### BACKGROUND OF THE INVENTION

1. Field of the Invention

The present invention relates to an apparatus and method for measuring a pulse wave, and more particularly, to an apparatus and method for measuring a pulse wave that can measure a pulse wave of a person to be examined in an unrestrictive state using a plurality of optical elements.

2. Description of the Related Art

In general, as a method that measures a photoplethysmogram (hereinafter, referred to as ‘PPG’) of a human being, optical sensors have been used. The method radiates light that reacts with blood flowing through blood vessels and converts the amount of reflected light or the amount of transmitted light into a signal, thereby measuring the PPG. This method using the optical sensors, which measures the PPG, is in contact with fingertips and earlobes of the human being. This is because portions of a body where capillary vessels are most developed are the fingertips and the earlobes of a human being and the PPG can be easiest measured at the corresponding portions.

The PPG has been widely used in various fields. For example, aging of blood vessels, a stress state analysis, and an emotional state analysis have been performed by measuring and analyzing the PPG. In recent years, with the advent of a ubiquitous environment, it has been required to measure the PPG to be suitable for the ubiquitous environment. One of the core characteristics of the ubiquitous environment is to support services in an unconscious state. However, in the existing PPG measuring methods, users are conscious of PPG measurement activities or the PPG is intentionally measured.

In the related art, in order to measure the PPG, measurement sensors that are made in a form of thimbles or nippers are worn on or engaged to fingertips or earlobes of the human being. In this case, since the user is restrained by the measurement apparatus, the user cannot perform daily tasks or function normally. Further, a cable that is connected to a measuring unit is exposed to the outside, regardless of the fingertip or the earlobe. The exposed cable causes inconvenience to the person being measured, which results in making it impossible for the measured person to measure the PPG while performing daily tasks. In order to remove the cable, a wireless communication circuit unit and a power supply circuit unit should be incorporated in a measuring unit circuit. In this case, however, the size of the measuring unit extraordinarily increases.

As described above, the methods according to the related art that are suggested to measure the PPG intentionally measure the PPG in the restrictive state. Accordingly, methods that measure the PPG in nonrestrictive state have been researched.

### SUMMARY OF THE INVENTION

Accordingly, it is an object of the present invention to provide an apparatus and method for measuring a pulse wave that can detect an optimal pulse wave signal according to combinations of a plurality of light emitting elements and a plurality of light receiving elements in an unrestrictive state.

In order to achieve the above objects, a pulse wave measuring apparatus according to the present invention is worn on a wrist of a person to be examined and measures a pulse wave signal. The pulse wave measuring apparatus comprises a sensor unit that includes a plurality of light emitting elements and a plurality of light receiving elements and detects a pulse wave signal of the person to be examined, and a control unit that selects any one of combinations of the plurality of light emitting elements and the plurality of light receiving elements according to signal sensitivity of a pulse wave signal that is detected for each combination, and activates the light emitting element and the light receiving element corresponding to the selected combination to measure a pulse wave.

The sensor unit includes the plurality of light emitting elements and the plurality of light receiving elements that are disposed in a line. Further, the sensor unit includes the plurality of light emitting elements and the plurality of light receiving elements that are alternately disposed. At this time, each of the combinations of the plurality of light emitting elements and the plurality of light receiving elements is composed of a pair of the light emitting element and the light receiving element that are adjacent to each other.

Meanwhile, the control unit includes a switching unit that controls an ON/OFF state of each of the plurality of light emitting elements and the plurality of light receiving elements. At this time, the switching unit turns on the corresponding light emitting element and light receiving element for each of the combinations of the plurality of light emitting elements and the plurality of light receiving elements. The control unit calculates an RMS of the pulse wave signal that is detected for each of the combinations of the plurality of light emitting elements and the plurality of light receiving elements, and measures signal sensitivity for the corresponding pulse wave signal.

Further, the pulse wave measuring apparatus according to the present invention further comprises a display unit that outputs pulse wave measurement data that is measured by the control unit.

In order to achieve the above objects, a pulse wave measuring method according to the present invention uses a pulse wave measuring apparatus that is worn on a wrist of a person to be examined and measures a pulse wave signal. The pulse wave measuring method comprises measuring signal sensitivity of a pulse wave signal that is detected for each of the combinations of a plurality of light emitting elements and a plurality of light receiving elements that are included in the sensor unit; selecting any one of the combinations on the basis of the measured signal sensitivity; and activating the light emitting element and the light receiving element corresponding to the selected combination and measuring the pulse wave.

The pulse wave signal in the measuring of the signal sensitivity is detected from the light emitting element and the light receiving element corresponding to each of the combinations, in a state where the plurality of light emitting elements and the plurality of light receiving elements are alternately disposed in a line. Further, each of the combinations of the plurality of light emitting elements and the plurality of light receiving elements is composed of a pair of the light emitting element and the light receiving element that are adjacent to each other.

Meanwhile, the measuring of the signal sensitivity includes controlling an ON/OFF state of each of the plurality of light emitting elements and the plurality of light receiving elements. At this time, the controlling of the ON/OFF state turns on only the light emitting element and light receiving element corresponding to the selected combination of the combinations of the plurality of light emitting elements and the plurality of light receiving elements.

The controlling of the ON/OFF state turns on the corresponding light emitting element and light receiving element for each of the combinations of the plurality of light emitting elements and the plurality of light receiving elements. Meanwhile, the measuring of the pulse wave, turns on only the light emitting element and the light receiving element corresponding to the combination that is selected in the selecting of the combination among the combinations of the plurality of light emitting elements and the plurality of light receiving elements.

The measuring of the signal sensitivity calculates an RMS of the pulse wave signal that is detected for each of the combinations of the plurality of light emitting elements and the plurality of light receiving elements, and measures signal sensitivity for the corresponding pulse wave signal.

The measuring of the signal sensitivity includes setting a maximum signal sensitivity value as zero, and updating the maximum signal sensitivity value to the signal sensitivity value measured for each of the combinations of the plurality of light emitting elements and the plurality of light receiving elements, when the measured signal sensitivity value is larger than the previous signal sensitivity value, for each of the combinations.

Meanwhile, the pulse wave measuring method according to the present invention further comprises outputting the pulse wave measurement data measured in the measuring of the pulse wave.

According to the present invention, since the pulse wave measuring apparatus is like a wristwatch or a wristband such that the pulse wave measuring apparatus can be worn on a wrist of a person to be examined, the pulse wave measuring apparatus can achieve superior portability and measure a pulse wave in an unrestrictive state.

Further, a pulse wave signal is detected for a combination of a plurality of light emitting elements and a plurality of light receiving elements in a state where the plurality of light emitting elements and the plurality of light receiving elements are alternately disposed in a line. Accordingly, an optimal pulse wave can be detected from a pulse wave signal that is detected by a combination of the light emitting element and the light receiving element that are adjacent to a blood vessel of a person to be examined.

Furthermore, stress, a health state, and an emotional state of the person to be examined can be periodically managed by transmitting pulse wave measured data of the person to be examined to an external apparatus.

### BRIEF DESCRIPTION OF THE DRAWINGS

FIG. 1 is a block diagram illustrating the configuration of a pulse wave measuring apparatus according to the present invention;

FIGS. 2A to 3 are exemplary views illustrating a pulse wave measuring apparatus according to an embodiment of the present invention;

FIGS. 4A and 4B are diagrams illustrating the configuration of a sensor unit in a pulse wave measuring apparatus according to an embodiment of the present invention;

FIGS. 5A to 7B are diagrams illustrating the operation of a sensor unit in a pulse wave measuring apparatus according to an embodiment of the present invention; and

FIG. 8 is a flowchart illustrating a flow of a pulse wave detecting method using a pulse wave measuring apparatus according to another embodiment of the present invention.

### DESCRIPTION OF THE PREFERRED EMBODIMENTS

Hereinafter, the preferred embodiments of the present invention will be described with reference to the accompanying drawings.

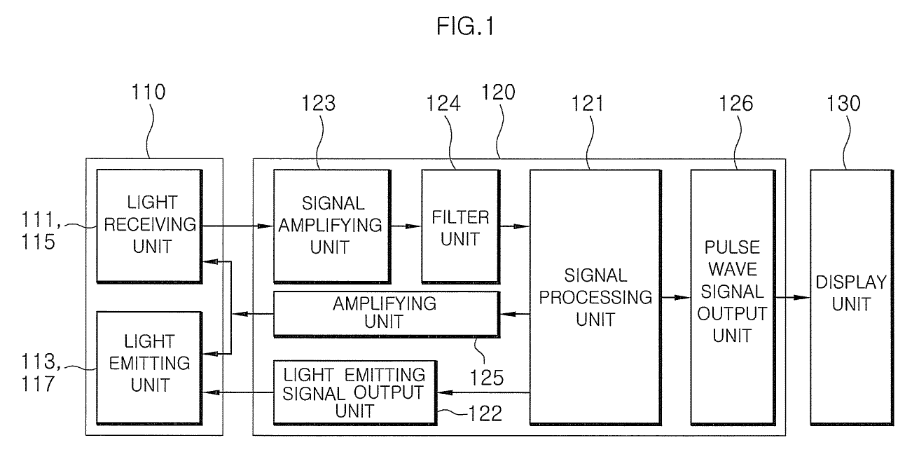

FIG. 1 is a block diagram illustrating the configuration of a pulse wave measuring apparatus according to the present invention. As shown in FIG. 1, the pulse wave measuring apparatus uses optical sensors to measure a photoplethysmogram (PPG) of a person to be examined. The pulse wave measuring apparatus includes a sensor unit 110, a control unit 120, and a display unit 130. In this case, the photoplethysmogram includes a heart rate, blood oxygen saturation, and contraction and expansion of blood vessels. Thus, a state of the person to be examined can be predicted through the photoplethysmogram.

First, the sensor unit 110 comes into contact with the skin of the person to be examined and detects a pulse wave signal of the corresponding portion. The sensor unit 110 includes a plurality of optical sensors, and the plurality of optical sensors are divided into a light emitting unit and a light receiving unit. In this case, the light emitting unit includes a plurality of light emitting elements 113 and 117 that emit optical signals, and light emitting diodes are used as the plurality of light emitting elements 113 and 117. At this time, the plurality of light emitting elements 113 and 117 are independently operated.

Meanwhile, the light receiving unit includes a plurality of light receiving elements 111 and 115 that receive the optical signals emitted from the plurality of light emitting elements 113 and 117, and photodiodes are used as the plurality of light receiving elements 111 and 115. At this time, the plurality of light receiving elements 111 and 115 are independently operated.

In this case, the sensor unit 110 is configured such that the plurality of light emitting elements 113 and 117 and the plurality of light receiving elements 111 and 115 are linearly disposed. At this time, the plurality of light emitting elements 113 and 117 and the plurality of light receiving elements 111 and 115 are alternately disposed. However, the present invention is not limited thereto, and the plurality of light emitting elements 113 and 117 and the plurality of light receiving elements 111 and 115 may be variously disposed other than the above. For example, the plurality of light emitting elements 113 and 117 and the plurality of light receiving elements 111 and 115 may be disposed in a zigzag type on a straight line.

Accordingly, the plurality of light emitting elements 113 and 117 and the plurality of light receiving elements 111 and 115 detect a pulse signal of the person to be examined at the places where the individual elements are located.

The control unit 120 is directly or indirectly connected to the sensor unit 110, and receives and processes the pulse wave signal that is detected by the sensor unit 110. At this time, the control unit 120 includes a signal processing unit 121, a light emitting signal output unit 122, a signal amplifying unit 123, a filter unit 124, a switching unit 125, and a pulse wave signal output unit 126.

The light emitting signal output unit 122 is connected to each of the plurality of light emitting elements 113 and 117, and adjusts the optical signals output from the light emitting elements. For example, the light emitting signal output unit 122 adjusts brightness of the optical signals that are output from the plurality of light emitting elements 113 and 117.

Meanwhile, the light receiving elements receive the optical signals that are output from the light emitting elements and apply the optical signals to the control unit 120. In this case, the optical signal that is received by the light receiving sensor is a signal that passes through the blood vessels of the person to be examined and is then reflected, and becomes the pulse wave signal.

The signal amplifying unit 123 is connected to each of the plurality of light receiving elements 111 and 115, and receives the pulse wave signal that is detected by each of the light receiving elements 111 and 115 and amplifies the pulse wave signal to have signal intensity of a predetermined value or more. The filter unit 124 removes a noise signal from the signal that is amplified by the signal amplifying unit 123. As such, the pulse wave signals that are detected by the light receiving elements 111 and 115 pass through the signal amplifying unit 123 and the filter unit 124 and are then input to the signal processing unit 121.

The signal processing unit 121 controls the operations of the light emitting signal output unit 122, the signal amplifying unit 123, the filter unit 124, the switching unit 125, and the pulse wave signal output unit 126. Further, the signal processing unit 121 reads the pulse wave signals that are detected by the plurality of light receiving elements 111 and 115 and measures signal sensitivity on the individual signals. For example, the signal processing unit 121 measures a root mean square (RMS) on the pulse wave signal that is detected by each of the plurality of light receiving elements 111 and 115, and measures signal sensitivity of the corresponding pulse wave signal from the measured RMS.

The signal processing unit 121 controls the operation of the switching unit 125, such that, for each of the combinations of the plurality of light emitting elements 113 and 117 and the plurality of light receiving elements 111 and 115, the light emitting element and the light receiving element corresponding to each combination are turned on, thereby measuring signal sensitivity of the pulse wave signal that is detected for each of the combinations. At this time, from the combinations of the plurality of light emitting elements 113 and 117 and the plurality of light receiving elements 111 and 115, the signal processing unit 121 selects a combination, from which the pulse wave signal having the largest signal sensitivity is detected.

The switching unit 125 is connected to the plurality of light emitting elements 113 and 117 and the plurality of light receiving elements 111 and 115, and controls an ON/OFF state of each of the plurality of light emitting elements 113 and 117 and the plurality of light receiving elements 111 and 115 in accordance with a control command from the signal processing unit 121. That is, when the signal processing unit 121 sequences the individual combinations of the plurality of light emitting elements 113 and 117 and the plurality of light receiving elements 111 and 115 and outputs a control command to activate the light emitting elements and the light receiving elements of the corresponding combinations in the predetermined sequence, the switching unit 125 turns on the corresponding elements in accordance with the control command from the signal processing unit 121, but turns off the other elements. At this time, the switching unit 125 continuously controls the ON/OFF states of the light emitting elements and the light receiving elements, until signal sensitivity measurement for each of the combinations is completed.

Further, if any one of the combinations is finally selected by the signal processing unit 121, the switching unit 125 finally turns on only the light emitting element and the light receiving element of the corresponding combination.

Accordingly, the signal processing unit 121 analyzes the pulse wave signal that is detected by the light emitting element and the light receiving element corresponding to the finally selected combination so as to measure a pulse wave, generates pulse wave measurement data including a pulse wave measurement result, and outputs the pulse wave measurement data to the outside through the pulse wave signal output unit 126. In this case, the pulse wave measurement data may include information of at least one of a frequency, a pulse wave velocity, and a pulse wave transit time, a pulse wave transit velocity of the corresponding pulse wave signal.

At this time, the pulse wave measurement data that is output from the pulse wave signal output unit 126 may be transmitted to an external apparatus using a wired or wireless communication method. For example, the pulse wave measuring apparatus is linked to a hospital information system, a medical portal, and a mobile communication apparatus, thereby realizing application scenarios, such as various health managements and disease managements. Further, the pulse wave measuring apparatus is linked to an intelligent robot, such that the robot can detect stress, a health state, and an emotional state through a pulse wave signal analysis of the human being. Accordingly, the pulse wave measuring apparatus can be used as a core element to implement various intelligent robot services.

The display unit 130 outputs the pulse wave measurement data, which is output from the pulse wave signal output unit 126, to a screen. In this case, examples of the display unit 130 may include generally used display units, such as an LCD, a PDP, and a touch screen. Of course, the pulse wave measurement data may be output through a voice output unit, such as a speaker, or other output units, although not shown in the drawings.

Meanwhile, the pulse wave measuring apparatus further includes a power supply unit (not shown) that supplies power to the sensor unit 110, the control unit 120, the display unit 130, and the internal elements.

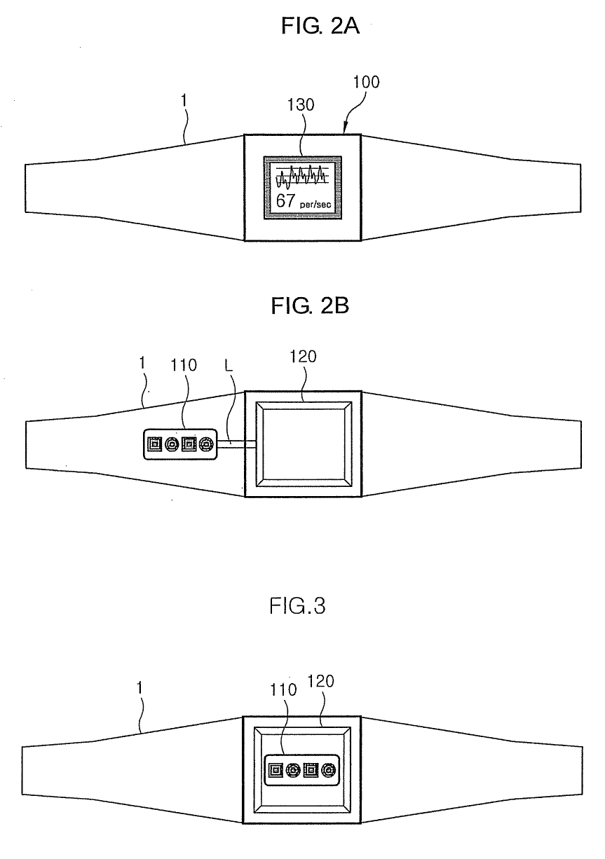

FIGS. 2A to 6 show an embodiment of a pulse wave measuring apparatus that is configured as shown in FIG. 1. In the embodiment of the present invention, the pulse wave measuring apparatus is implemented to be mounted in a band, but the present invention is not limited thereto.

FIGS. 2A and 2B show a front surface and a rear surface of a band on which a pulse wave measuring apparatus is mounted. FIG. 2A shows a front surface of a band 1, and FIG. 2B shows a rear surface of the band 1. At this time, it is assumed that the rear surface directly contacts a wrist of the person to be examined.

As shown in FIG. 2A, a body 100 of the pulse wave measuring apparatus is provided at a center of the band 1 that can be worn on the wrist of the person to be examined, and the display unit 130 that outputs the pulse wave signal is provided on the front surface of the band 1. As shown in FIG. 2B, the control unit 120 that is mounted on the body 100 of the pulse wave measuring apparatus and the sensor unit 110 that directly contacts the skin of the person to be examined and detects the pulse wave signal from the blood vessels are provided on the rear surface of the band 1. At this time, the sensor unit 110 is disposed to be away from the control unit 120, is connected to the control unit 120 through a connecting line L, and applies a detection signal to the control unit 120 through the connecting line L.

In this case, the sensor unit 110 is disposed to be away from the control unit 120 at a left side or a right side, and the sensor unit 110 and the control unit 120 are connected to each other through the connecting line L. At this time, the connecting line L includes a signal line through which the detected pulse wave signal is transmitted and a power line that that supplies operation power.

FIG. 3 shows another embodiment of the configuration that is shown in FIG. 2B. In this case, the sensor unit 110 is directly connected to the control unit 120. Since the sensor unit 110 is directly connected to the control unit 120, a separate connecting line is not needed.

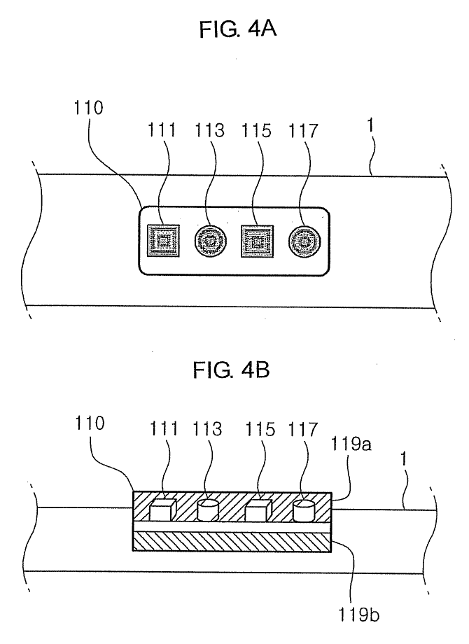

FIGS. 4A and 4B are diagrams illustrating the configuration of a sensor unit 110 according to the present invention. FIG. 4A is a front view of a sensor unit 110, and FIG. 4B is a lateral cross-sectional view of the sensor unit 110.

The sensor unit 110 has the configuration where the plurality of light emitting elements 113 and 117 and the plurality of light receiving elements 111 and 115 are disposed linearly on a substrate. At this time, since the thickness of a wrist and a location of a vein are different in each person to be examined, the substrate is formed of thin material that can ensure flexibility. Accordingly, since the plurality of light emitting elements 113 and 117 and the plurality of light receiving elements 111 and 115 come into surface contact with the wrist of the person to be examined, it is possible to detect a pulse wave signal according to each location.

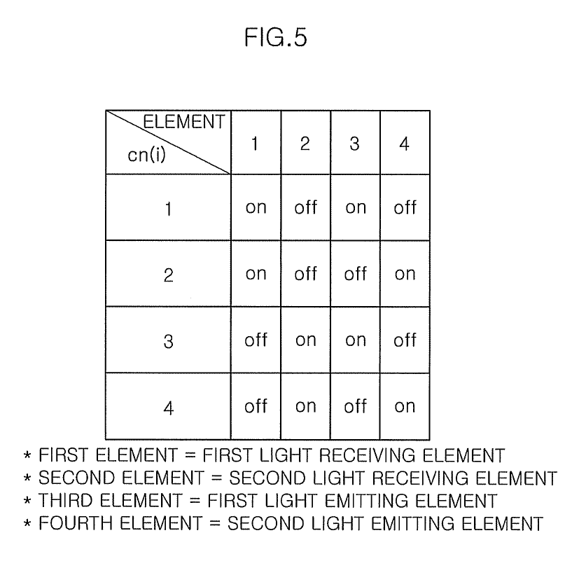

Further, since the location of the blood vessel is different for each person to be examined, if the plurality of light emitting elements 113 and 117 and the plurality of light receiving elements 111 and 115 are alternately disposed in a line, it is possible to accurately measure a pulse wave signal using the light emitting elements and the light receiving elements at the places where the blood vessels are located. This embodiment is shown in FIG. 5.

Further, material, such as silicon or epoxy, which is soft, has excellent elasticity, and prevents light from being transmitted, is attached to top and bottom surfaces of a substrate where the light emitting elements 113 and 117 and the light receiving element 111 and 115 are attached. At this time, portions of the light emitting elements 113 and 117 and the light receiving elements 111 and 115 of the sensor unit 110 are opened on the top surface of the substrate, such that a pulse wave signal can be detected. Accordingly, when a pulse wave of the person to be examined is measured, the pulse wave signal is detected using the optical sensors. Thus, elements of light, except for an optical signal between the light emitting elements 113 and 117 and the light receiving elements 111 and 115, are blocked.

Meanwhile, when the sensor unit 110 is attached to the band 1, a groove that has the same size as the sensor unit 110 is formed in a predetermined area of the band 1, such that the sensor unit 110 is inserted into the groove. Accordingly, when the sensor unit 110 contacts the skin of the person to be examined, it is possible to prevent light from being incident from the side.

FIGS. 5 to 7B show an embodiment of a sensor unit.

FIG. 5 shows an element combination table for light emitting elements and light receiving elements that are included in a sensor unit. In this case, ON/OFF states of the light emitting elements and the light receiving elements are shown for each combination. At this time, in order to promote understanding of the embodiment of the present invention, a channel number is assigned to a signal that is detected for each combination.

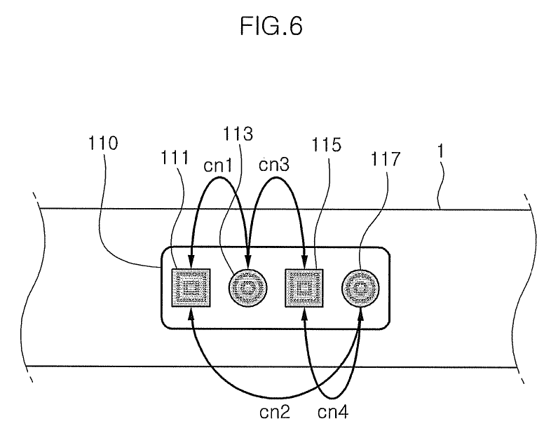

FIG. 6 shows a signal channel that is transmitted and received between a plurality of light emitting elements and a plurality of light receiving elements. In this case, each element combination is shown in the element combination table shown in FIG. 5.

The locations of blood vessels that can detect an optimal pulse wave signal for an individual person are various, and the optical pulse signal can be detected through the combinations of the plurality of light emitting elements and the plurality of light receiving elements. In this embodiment, four elements, that is, the two light receiving elements 111 and 115 and the two light emitting elements 113 and 117 are provided. However, the number of each of the light emitting elements and the light receiving elements may be changed according to the sizes and types of the elements.

The four elements that are shown in FIG. 6 are referred to as a first element, a second element, a third element, and a fourth element, for convenience of explanation. In this case, it is assumed that the first element is the first light receiving element 111, the second element is the second light receiving element 115, the third element is the first light emitting element 113, and the fourth element is the second light emitting element 117.

At this time, the light receiving elements 111 and 115 and the light emitting elements 113 and 117 are alternately disposed in a straight line, thereby forming a total of four combinations. That is, when the elements are disposed in the order of the first element 111, the third element 113, the second element 115, and the fourth element 117, combinations of {the first element and the third elements}, {the first element and the fourth element}, {the second element and the third element}, and {the second element and the fourth element} are formed. At this time, a signal that is measured by the combination of {the first element and the third element} is referred to as a first channel cn1, a signal that is measured by the combination of {the first element and the fourth element} is referred to as a second channel cn2, a signal that is measured by the combination of {the second element and the third element} is referred to as a third channel cn3, and a signal that is measured by the combination of {the second element and the fourth element} is referred to as a fourth channel cn4.

Accordingly, the signal processing unit 121 measures signal sensitivity for each of the four channels cn1, cn2, cn3, and cn4 that are measured by the individual signal combinations, confirms the channel having optimal signal sensitivity, and measures a pulse wave from a pulse wave signal that is detected by activating only an element corresponding to the relevant channel.

In the embodiment shown in FIG. 6, all of the combinations are formed by the pairs of the light emitting elements and the light receiving elements using the two light emitting elements and the two light receiving elements. However, in some case, the combinations may be formed by only the pairs of the light emitting elements and the light receiving elements adjacent to each other. For example, since the third element 113 is adjacent to the first element 111 and the second element 115, combinations of {the first element and the third element} and {the second element and the third element} are formed. Since the fourth element 117 is adjacent to the second element 115, a combination of {the second element and the fourth element} is formed. Accordingly, since the first element 111 and the fourth element 117 are not adjacent to each other, signal sensitivity is measured to be lower than the other combinations. Thus, the corresponding combination is not formed, or when the corresponding combination is already formed, the pulse wave signal is not detected.

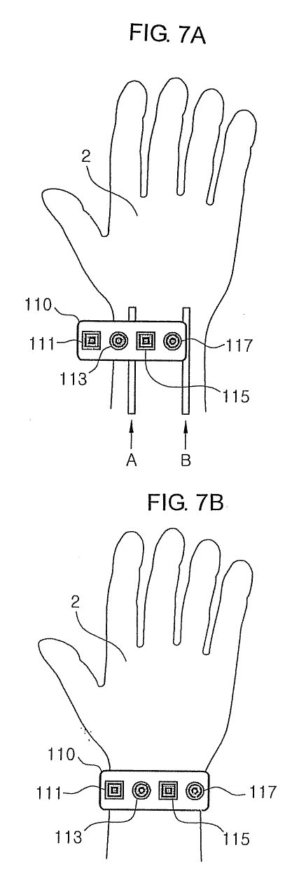

FIGS. 7A and 7B show an embodiment according to a location of a sensor unit in a pulse wave measuring apparatus that is mounted on a wrist of a person to be examined. FIG. 7A shows the case where a sensor unit 110 is located to be biased to the left side of a wrist. In this case, the sensor unit 110 detects a pulse wave signal from a radial artery ‘A’ in blood vessels A and B of the person to be examined. Accordingly, the signal processing unit 121 measures a pulse wave through the third channel cn3 according to a combination of {the second element and the third element} that are located around the radial artery ‘A’.

FIG. 7B shows the case where a sensor unit is located at the center of a wrist of a person to be examined. In this case, different from FIG. 7A, the signal processing unit 121 measures a pulse wave through the first channel cn1 according to a combination of {the first element and the third element} that are located around the radial artery ‘A’.

The operation of the present invention that has the above-described configuration is as follows.

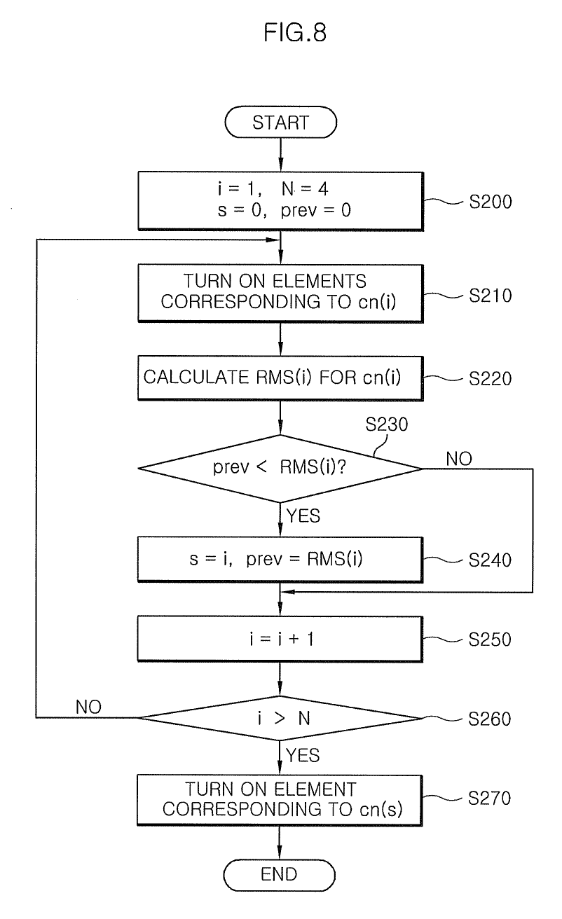

FIG. 8 is a flowchart illustrating an operational flow of a pulse wave measuring apparatus according to an embodiment of the present invention, which shows a process of detecting an optimal pulse wave signal according to a plurality of element combinations. At this time, the entire operational flow of the pulse wave measuring apparatus is omitted.

In the pulse wave measuring apparatus according to the embodiment of the present invention, when a power supply device is turned on, an optical signal is output from a light emitting unit of the sensor unit 110 as a control signal is output from the light emitting signal output unit 122. At this time, the optical signal that is output from the light emitting unit passes through a blood vessel of the skin of the person to be examined and is then input to the light receiving unit.

At this time, the signal that is input to the light receiving unit as a pulse wave signal is output such that a signal frequency is different according to a bloodstream speed of the blood vessels. The pulse wave signal that is input to the light receiving unit is transmitted to a signal amplifying unit 123 of the control unit 120, and the signal amplifying unit 123 amplifies the input pulse wave signal as a signal having a predetermined value. The partial noise of the pulse wave signal that is amplified by the signal amplifying unit 123 is removed by the filter unit 124 and the pulse wave signal is transmitted to the signal processing unit 121.

At this time, the signal processing unit 121 detects an optimal pulse wave signal according to an element combination of the sensor unit 110, and the detected pulse wave signal is output to the outside through the pulse wave signal output unit 126. The pulse wave signal that is output through the pulse wave signal output unit 126 is transmitted to the display unit 130 and output to a screen, and may be transmitted to an external apparatus using a wired or wireless communication method.

In this case, the process of detecting an optimal pulse wave signal is described with reference to FIG. 8. The signal processing unit 121 controls the operation of a switching unit 125 to turn on the elements (the first and second elements) corresponding to the first channel cn(1) (Step S210). Of course, initial control variables according to the driving of the pulse wave measuring apparatus are set as a channel number i=1, the number of channels N=4, a selection channel s=0, and signal sensitivity prev=0 (S200). Of course, an initial variable value for prev may be changed according to setting.

At this time, the signal processing unit 121 calculates RMS(1) of the first channel cn(1) detected from the combination of {the first element and the third element}, thereby measuring signal sensitivity for cn(1) (S220). In a process of ‘S230’, since RMS(1) has a higher value than ‘0’ that is an initial value of prev, s=1 and prev=RMS(1) are obtained in a process of ‘S240’.

Further, the signal processing unit 121 increases a value of ‘i’ by 1 (S250) and allows the elements (the first element and the fourth element) corresponding to cn(2) to be turned on (S210). At this time, the signal processing unit 121 calculates RMS(2) of cn(2) that is detected by the combination of {the first element and the fourth element}, thereby measuring signal sensitivity for cn(2) (S220). In a process of ‘S230’, RMS(2) and RMS(2) as the current prev value are compared and a high value is set as the prev value. That is, when a value of signal sensitivity for cn(2) is larger than a previous value of signal sensitivity, a maximum signal sensitivity value is updated as a current signal sensitivity value.

When RMS(1) is larger than RMS(2), the process of ‘S240’ is omitted and the value of ‘i’ is immediately increased by ‘1’ (S250), such that the elements (the second element and the third element) corresponding to cn(3) are turned on (S210). Meanwhile, when RMS(1) is smaller than RMS(2), in the process of ‘S240’, s=2 and prev=RMS(2) are obtained. Then, the signal processing unit 121 increases the value of ‘i’ by ‘1’ (S250), such that the elements (the second element and the third element) corresponding to cn(3) are turned on (S210).

Like this, the signal processing unit 121 calculates RMS(3) of cn(3) that is detected from the combination of {the second element and the third element}, thereby allowing signal sensitivity for cn(3) to be measured (S220). In the process of ‘S230’, RMS(3) and the prev value are compared and a high value is set as the prev value. When the RMS(3) is larger than the current prev value, in the process of ‘S240’, s=3 and prev=RMS(3) are obtained. Then, the signal processing unit 121 increases the value of ‘i’ by ‘1’ (S250), such that the elements (the third element and the fourth element) corresponding to cn(4) are turned on (S210). Meanwhile, when RMS(3) is smaller than the current prev value, the process of ‘S240’ is omitted and the value of ‘i’ is immediately increased by ‘1’ (S250), such that the elements (the third element and the fourth element) corresponding to cn(4) are turned on (S210).

Finally, the signal processing unit 121 calculates RMS(4) of cn(4) that is detected from the combination of {the third element and the fourth element}, thereby allowing signal sensitivity for cn(4) to be measured (S220). In the process of ‘S230’, RMS(4) and the prev value are compared and a high value is set as the prev value. When the RMS(4) is larger than the current prev value, in the process of ‘S240’, s=4 and prev=RMS(4) are obtained. Meanwhile, when RMS(4) is smaller than the current prev value, the process of ‘S240’ is omitted.

At this time, in the process of ‘S260’, since there is no signal channel that is needed to measure signal sensitivity, the signal processing unit 121 selects a signal channel having the highest signal sensitivity, that is, cn(2) on the basis of the final s value and prev value, and turns on only the elements corresponding to the selected cn(s), thereby detecting the optimal pulse wave signal (S270).

Accordingly, the pulse wave measuring apparatus according to the embodiment of the present invention detects the pulse wave signal for each of the combinations of the plurality of light emitting elements and the plurality of light receiving elements, thereby measuring the optimal pulse wave. Accordingly, it is possible to provide the accurate pulse wave measured data to the person to be examined.

The apparatus and method for measuring a pulse wave according to the embodiment of the present invention are limited to the embodiments. All or a portion of the embodiments may be configured to be selectively combined, such that various modifications and changes can be made.

What is claimed is:

1. A pulse wave measuring apparatus that is worn on a wrist of a person to be examined and measures a pulse wave signal, comprising:
a sensor unit that includes a plurality of light emitting elements and a plurality of light receiving elements and detects a pulse wave signal of the person to be examined; and
a control unit that selects any one of combinations of the plurality of light emitting elements and the plurality of light receiving elements according to signal sensitivity of a pulse wave signal that is detected for each combination, and activates the light emitting element and the light receiving element corresponding to the selected combination to measure a pulse wave.

2. The pulse wave measuring apparatus of claim 1,
wherein the sensor unit includes the plurality of light emitting elements and the plurality of light receiving elements that are disposed in a line.

3. The pulse wave measuring apparatus of claim 1,
wherein the sensor unit includes the plurality of light emitting elements and the plurality of light receiving elements that are alternately disposed.

4. The pulse wave measuring apparatus of claim 1,
wherein each of the combinations of the plurality of light emitting elements and the plurality of light receiving elements is composed of a pair of the light emitting element and the light receiving element that are adjacent to each other.

5. The pulse wave measuring apparatus of claim 1,
wherein the control unit includes a switching unit that controls an ON/OFF state of each of the plurality of light emitting elements and the plurality of light receiving elements.

6. The pulse wave measuring apparatus of claim 5,
wherein the switching unit turns on the corresponding light emitting element and light receiving element for each of the combinations of the plurality of light emitting elements and the plurality of light receiving elements.

7. The pulse wave measuring apparatus of claim 1,
wherein the control unit calculates an RMS of the pulse wave signal that is detected for each of the combinations of the plurality of light emitting elements and the plurality of light receiving elements, and measures signal sensitivity for the corresponding pulse wave signal.

8. The pulse wave measuring apparatus of claim 1, further comprising:
a display unit that outputs pulse wave measurement data that is measured by the control unit.

9. A pulse wave measuring method of a pulse wave measuring apparatus that is worn on a wrist of a person to be examined and measures a pulse wave signal, comprising:
measuring signal sensitivity of a pulse wave signal that is detected for each of the combinations of a plurality of light emitting elements and a plurality of light receiving elements that are included in the sensor unit;
selecting any one of the combinations on the basis of the measured signal sensitivity; and
activating the light emitting element and the light receiving element corresponding to the selected combination and measuring the pulse wave.

10. The pulse wave measuring method of claim 9,
wherein the pulse wave signal in the measuring of the signal sensitivity is detected from the light emitting element and the light receiving element corresponding to each of the combinations, in a state where the plurality of light emitting elements and the plurality of light receiving elements are alternately disposed in a line.

11. The pulse wave measuring method of claim 9,
wherein each of the combinations of the plurality of light emitting elements and the plurality of light receiving elements is composed of a pair of the light emitting element and the light receiving element that are adjacent to each other.

12. The pulse wave measuring method of claim 9,
wherein the measuring of the signal sensitivity includes:
controlling an ON/OFF state of each of the plurality of light emitting elements and the plurality of light receiving elements.

13. The pulse wave measuring method of claim 12,
wherein, the controlling of the ON/OFF state turns on the corresponding light emitting element and light receiving element for each of the combinations of the plurality of light emitting elements and the plurality of light receiving elements.

14. The pulse wave measuring method of claim 9,
wherein, in the measuring of the pulse wave turns on only the light emitting element and the light receiving element corresponding to the combination that is selected in the selecting of the combination among the combinations of the plurality of light emitting elements and the plurality of light receiving elements.

15. The pulse wave measuring method of claim 9,
wherein, the measuring of the signal sensitivity, calculates an RMS of the pulse wave signal that is detected for each of the combinations of the plurality of light emitting elements and the plurality of light receiving elements, and measures signal sensitivity for the corresponding pulse wave signal.

16. The pulse wave measuring method of claim 9,
wherein the measuring of the signal sensitivity includes:
setting a maximum signal sensitivity value as zero; and
updating the maximum signal sensitivity value to the signal sensitivity value measured for each of the combinations of the plurality of light emitting elements and the plurality of light receiving elements, when the measured signal sensitivity value is larger than the previous signal sensitivity value, for each of the combinations.

17. The pulse wave measuring method of claim 9, further comprising:
outputting the pulse wave measurement data measured in the measuring of the pulse wave.
